(19) United States
(12) Patent Application Publication
(10) Pub. No.: US 2024/0180998 A1
(43) Pub. Date: Jun. 6, 2024

(54) MULTILAYERED PATCH

(71) Applicant: KØBENHAVNS UNIVERSITET, København K (DK)

(72) Inventors: Mai Bay STIE, København K (DK); Heidi ÖBLOM, København K (DK); Jette JACOBSEN, København K (DK); Jukka RANTANEN, København K (DK); Hanne Mørck NIELSEN, København K (DK); Natalja GENINA, København K (DK); Cristiana Filipa BARREIRO DA CUNHA, København K (DK)

(73) Assignee: KØBENHAVNS UNIVERSITET, København K (DK)

(21) Appl. No.: 18/553,048

(22) PCT Filed: Apr. 6, 2022
(86) PCT No.: PCT/EP2022/059128

§ 371 Date: Sep. 28, 2023

(30) Foreign Application Priority Data
Apr. 7, 2021 (EP) 21167196.1

(51) Int. Cl. A61K 38/095; A61J 7/00; A61K 9/00; A61K 31/4174; A61K 31/525; A61K 31/721; A61K 47/34; A61K 47/36; A61K 47/38

(52) U.S. Cl. CPC A61K 38/095; A61J 7/0053; A61K 9/006; A61K 31/4174; A61K 31/525; A61K 31/721; A61K 47/34; A61K 47/36; A61K 47/38

(57) ABSTRACT

A dosage form for application on mucosae for release of at least one active pharmaceutical ingredient, wherein the dosage form includes an adhesive layer, made of fibers, capable of adhering to the mucosae of the human or animal body, a water-repelling backing layer, and at least one intermediate layer positioned between the adhesive layer and the water-repelling backing layer. The active pharmaceutical ingredient(s) are incorporated in the intermediate layer(s) and/or in the adhesive layer.

### TECHNICAL FIELD

The present invention relates to a dosage form for application on mucosae for release of at least one active pharmaceutical ingredient.

### BACKGROUND

The oral route is the most common and accepted route for administration of drugs, most often in the form of tablets and capsules. However, due to swallowing difficulties, up to about 60% of patients modify their oral intake of drugs in a way that may alter safety and efficacy, and up to about 10% are non-adherent to their treatment (Schiele et al., 2013, European Journal of Clinical Pharmacology, 69(4), 937-948). Furthermore, drug absorption and sufficient systemic bioavailability of some drugs are often hindered after oral intake due to degradation in the harsh conditions of the gastrointestinal tract, i.e. the low pH of the stomach and the presence of digestive enzymes in the intestines. Dosage forms that are applied on the oral mucosa avoid the harsh conditions in the gastrointestinal tract, can be taken without water and do not require swallowing of the drug or the dosage form. This can be beneficial for patients suffering from e.g. dysphagia. Current dosage forms intended for such oromucosal administration include, amongst others, oral films and disintegrating tablets used for systemic as well as local drug delivery. The films are commonly prepared by solvent casting, semi-solid casting, hot-melt extrusion (HME), solid-dispersion extrusion, or rolling, whereas tablets are usually prepared by compression and compaction or freeze-drying (lyophilisation). However, these dosage forms struggle from short residence time in the oral cavity and significant washout of the drug as well as the entire dosage form by saliva. This limits drug absorption in the oral cavity and can lead to low drug bioavailability and unwanted side effects upon swallowing. Higher doses are often needed to be incorporated in these oromucosal dosage forms to guarantee a therapeutic effect and many of the current dosage forms suffer from the drawback that they do not allow for high drug loadings. Further, these dosage forms can be sensitive to ambient conditions e.g. they become extremely brittle in a dry environment, and lose their physical structure (disintegrate and/or dissolve) in humid conditions. In addition, mechanical properties, such as flexibility of the films and strength of the dosage forms, are not always optimal for manufacturing and handling by the patient. The current dosage forms such as films are difficult to handle, especially for elderly people with physiological impairments (e.g. tremor). Furthermore, incorporation of more than one active pharmaceutical ingredient with a tailored (immediate or controlled) release profile for each active pharmaceutical ingredient in a single dosage form is challenging with the current dosage forms. In general, patch technologies are well suitable for topical application, because of their ease of use and large surface area.

Patches based on fibers have been developed for local and systemic delivery of active pharmaceutical ingredients. However, for these patches, the active pharmaceutical ingredient(s) are loaded in a layer composed of fibers most often prepared by the electrospinning technique. Not all active pharmaceutical ingredients are compatible with the electrospinning technique and the loading capacity in fibers can be a limiting factor. Furthermore, loading of multiple active pharmaceutical ingredients and/or with specific excipients within the same dosage form by the electrospinning technique alone can be a significant challenge as the parameters for successful fiber preparation by electrospinning of each of the components may not be compatible. Having multiple layers and several methods for their preparation, expands the versatility and potential usability of a dosage form of multiple layers.

Thus, in view of the above there is still a need for dosage forms for mucosal application for efficient drug delivery; dosage forms, which are safe and easily applied by the patients, for versatile drug loading and release, as well as feasible to manufacture in large scale.

It is the objective of at least certain aspects of the present invention to provide an improvement over the current techniques and systems of known art, some of which are described above, and particularly, to achieve a dosage form that is easy to apply to a human or animal body and that ensures controlled and unidirectional release of sufficient amounts of at least one active pharmaceutical ingredient.

### SUMMARY OF THE INVENTION

Accordingly, a first aspect of the present invention provides a dosage form for application to mucosa for release of at least one active pharmaceutical ingredient comprising an adhesive layer capable of adhering to mucosa of the human or animal body; a water-repelling backing layer; and at least one intermediate layer positioned between the adhesive layer and the water-repelling backing layer; wherein the at least one active pharmaceutical ingredient is incorporated in the at least one intermediate layer and/or in the adhesive layer, and wherein the at least one intermediate layer is a porous semi-solid or solid material and the adhesive layer is made of fibres.

Close connection between the individual layers of the invention provide a single dosage unit that can be handled, stored, marketed, distributed, and administered as such. Hence, according to any aspect, the individual layers in the dosage unit are placed on top of each other in close connection.

By the present invention is achieved that at least one active pharmaceutical ingredient can be loaded in each of the individual layers, most preferably in the intermediate layer or the adhesive layer. This can be done by one of multiple techniques, i.e., electrospinning, freeze-drying or spraying, giving the current invention a great versatility, both in terms of the drug types that can be loaded, the loading capacity but also the tuneability of the release profile.

Hence, the at least one active pharmaceutical ingredient may be incorporated in the at least one intermediate layer and/or in the adhesive layer.

According to any aspects, there may be one intermediate layer where the active pharmaceutical ingredient may be incorporated in.

According to any aspects, the active pharmaceutical ingredient may be incorporated in the adhesive layer.

According to any aspects, there may be one intermediate layer and one adhesive layer, where the active pharmaceutical ingredient may be incorporated in both the intermediate layer and in the adhesive layer.

According to any aspects, there may be one intermediate layer with one active pharmaceutical ingredient incorporated therein and one adhesive layer, with another different active pharmaceutical ingredient incorporated therein.

According to any aspects, there may be two intermediate layers. In one embodiment, there may be three intermediate layers. An active pharmaceutical ingredient may be incorporated in either, and potentially all of the intermediate layers. The active pharmaceutical ingredient may be the same in all the intermediate layers or it may be different active pharmaceutical ingredients in the individual intermediate layers.

According to any aspects, an active pharmaceutical ingredient may be incorporated in all of the intermediate layers and also in the adhesive layer. The active pharmaceutical ingredient may be the same in all the intermediate layers and in the adhesive layer or it may be different active pharmaceutical ingredients in the individual layers.

The present multi-layered dosage form thus being a significant benefit over existing patches based only on nanofibers or semi-solid or solid materials, e.g., foams.

Having multiple layers and several methods for their preparation thus not only electrospinning or freeze-drying or spraying, expands the versatility and potential usability of a dosage form of multiple layers by enabling the loading of a variety of drug types and excipients alone or together.

According to any aspects, the at least one intermediate layer is a freeze dried or vacuum dried intermediate layer.

According to any aspects, the at least one intermediate layer is a foam layer, e.g. a solid foam layer.

According to any aspects, the adhesive layer is an adhesive fibre layer.

According to any aspects, the at least one intermediate layer is a solid foam layer and the adhesive layer is an adhesive fibre layer and the backing layer is a water-repelling layer.

According to one aspect, the dosage form comprises:
an adhesive fibre layer;
a water-repelling backing layer; and
one solid foam intermediate layer comprising the at least one active pharmaceutical ingredient.

According to one aspect, the dosage form comprises:
an adhesive fibre layer;
a water-repelling backing layer; and
one freeze-dried intermediate layer comprising the at least one active pharmaceutical ingredient.

According to one aspect, the dosage form comprises:
an adhesive fibre layer;
a water-repelling backing layer; and
two or more solid foam intermediate layers each comprising the at least one active pharmaceutical ingredient.

According to one aspect, the dosage form comprises:
an adhesive fibre layer;
a water-repelling backing layer; and
two or more freeze-dried intermediate layers each comprising the at least one active pharmaceutical ingredient.

According to one aspect, the dosage form comprises:
an adhesive fibre layer comprising the at least one active pharmaceutical ingredient;
a water-repelling backing layer; and
at least one solid foam intermediate layer.

According to one aspect, the dosage form comprises:
an adhesive fibre layer comprising the at least one active pharmaceutical ingredient;
a water-repelling backing layer; and
at least one freeze-dried intermediate layer.

Freeze-drying, in particular, benefits from limited loss of material and low drying temperatures, which is beneficial for encapsulation of labile active pharmaceutical ingredients. Furthermore, it is a very versatile technique, thus providing a great versatility in terms of loading capacity of different active pharmaceutical ingredients.

Fibers prepared by electrospinning have shown great adhesive properties to the mucosa and are able to modify the release rate of active pharmaceutical ingredients from the dosage form.

The dosage form for application to mucosa may be a patch.

According to one aspect, the mucosa may be selected from the group consisting of oral cavity, digestive tract, nasal, rectal, ocular and/or vaginal mucosae.

According to any aspect, mucosa may be healthy or diseased, intact or compromised.

A second aspect of the present invention provides a method of forming the dosage form according to the present invention, comprising the steps of forming the at least one intermediate layer, covering the intermediate layer from one side with an adhesive layer which is capable of adhering to the mucosa of a human or animal body, and covering the at least one intermediate layer from the other side with a water-repelling backing layer.

The method may be done by batch or continuous processing.

During the method, the at least one active pharmaceutical ingredient can be incorporated in the at least one intermediate layer and/or in the adhesive layer.

In one aspect, the at least one intermediate layer is comprising the at least one active pharmaceutical ingredient.

According to any aspect, the porous semi-solid or solid material of the intermediate layer(s) may be prepared by freeze-drying or vacuum drying.

According to any aspect, the adhesive layer may be formed by spinning.

According to any aspect, the covering of the intermediate layer(s) with a water-repelling backing layer may be done by spraying or casting the water-repelling backing layer onto the intermediate layer, by freeze-drying of the porous semi-solid or solid material onto the backing layer, or by applying a heated preformed backing layer onto the intermediate layer or chemically by wetting the surface of the layer(s) with the appropriate solvent and attaching them together as one unit.

According to any aspect, the porous semi-solid or solid material may be formed by freeze-drying or vacuum drying of an aqueous or organic polymer dispersion.

According to any aspect, the intermediate layer(s), the adhesive layer and the water-repelling backing layer may comprise one or more hydrophilic and/or hydrophobic polymers and/or gums.

According to any aspect, said one or more hydrophilic and/or hydrophobic polymer and/or gums may be selected from the group consisting of polyethylene oxide, methyl cellulose, hydroxypropyl cellulose, hydroxyethyl cellulose, carboxymethyl cellulose, methylhydroxyethylcellulose, hydroxypropyl methylcellulose, hydroxyethylcarboxymethylcellulose, carboxymethylhydroxyethylcellulose, polyvinylpyrrolidone, ethylcellulose chitosan, polyacrylic acid, polylactic acid, poly(lactic-co-glycolic acid), ethylcellulose, acacia, gellan gum, gelatin and polysaccharides, such as xanthan, agar, guar gum, carrageenans, carrageenan agar, alginic acid, polymethacrylate, and/or mixtures, and/or salts and/or derivatives hereof.

According to any aspect, the adhesive layer may comprise at least one adhesive polymer, comprising chitosan, alginate, polyethylene oxide and/or salt and/or derivatives hereof.

According to any aspect, the intermediate layer may comprise of methyl cellulose, hydroxypropyl cellulose, hydroxyethyl cellulose, carboxymethyl cellulose, hydroxypropyl methylcellulose, xanthan, agar, guar gum, alginic acid, polymethacrylate, and/or mixtures, and/or salts and/or derivatives hereof.

According to any aspect, the water-repelling backing layer may comprise of polyvinylpyrrolidone, ethylcellulose, polymethacrylates and/or mixtures and/or salt and/or derivatives hereof.

According to any aspect, the one or more hydrophilic and/or hydrophobic polymer and/or gums may be in an aqueous or organic dispersion, or neat polymer, with/before fabrication of layers by e.g. spraying, lyophilisation, spinning, casting, or drying.

According to any aspect, the adhesive layer may be produced by electrospinning, wet spinning, dry spinning or melt spinning.

According to any aspect, the intermediate layer may be produced by freeze-drying or vacuum drying.

According to any aspect, the water-repelling backing layer may be produced by spraying or casting a polymer dispersion and/or by film formation by heating or light irradiation or by adhering a preformed water-repelling backing layer by wetting or heat.

According to any aspect, the fibres may be produced from an aqueous polymer dispersion and/or an organic polymer dispersion or neat polymer.

According to any aspect, the adhesive layer may comprise supporting polymers to enable the formation of fibers by, e.g., electrospinning and may additionally contain one or more excipients. The supporting polymer(s) may comprise polyethylene oxide or polyvinyl alcohol.

According to any aspect, the excipients may consist of compound(s) that enable feasible preparation of the adhesive layer and/or has functionalities that enable enhanced drug absorption. The excipient may be selected from the group comprising (physically, chemically or microbially) stabilizing agents, pH adjusting agents, osmolality adjusting agents, membrane modulating agents, and moisture modulating agents.

According to any aspect, the layers may comprise plasticizers to improve the mechanical properties of the dosage form to allow the dosage form to be applied to curved surfaces such as mucosa.

According to any aspect, the dosage form may comprise two or more intermediate layers, and each intermediate layer may comprise at least one active pharmaceutical ingredient.

The adhesive layer may or may not comprise an active pharmaceutical ingredient.

According to any aspect, said at least one active pharmaceutical ingredient may be incorporated in the intermediate layer(s) and/or in the adhesive layer, and wherein the intermediate layer(s) may be a porous semi-solid or solid material, and the adhesive layer comprises fibres.

According to any aspect, the active pharmaceutical ingredient may be selected from one or more of the groups consisting of small molecular entities, including lipids; and peptides, proteins, and oligonucleotides, whether prepared by synthesis or expression and/or extraction; and biological material, such as living cells; and particulate matter.

According to any aspect, the active pharmaceutical ingredient may comprise anti-inflammatory compounds, hormones, cannabinoids, opioids, immunosuppressive compounds, immune stimulating compounds e.g. vaccines, antibiotics, antivirals, antifungal, chemotherapeutics, probiotics, prebiotics, nutrients, vitamins, minerals, trace elements, and combinations hereof.

According to any aspect, the active pharmaceutical ingredient may be present as dissolved, undissolved (droplets, amorphous or crystalline) or partly dissolved.

A third aspect of the present invention provides a dosage form for use in administration of an active pharmaceutical ingredient to a human or animal, wherein the adhesive layer adheres to mucosa, and wherein the active pharmaceutical ingredient is released unidirectionally in a controlled way.

At least some of the above identified and other objectives and advantages that may be apparent from the description have been achieved by the present dosage form and the present method.

### BRIEF DESCRIPTION OF THE DRAWINGS

These and other aspects, features and advantages of which embodiments of the invention are capable of, will be apparent and elucidated from the following description of embodiments and aspects of the present invention, reference being made to the accompanying drawings, in which

FIG. 1 is a schematic representation of the concept for the multi-layered drug delivery system (DDS) based on i) an intermediate layer consisting of a porous solid material (foam) for drug loading, acceptable physical stability, and mechanical properties, ii) an adhesive electrospun nanofiber layer with adhesive properties, for prevention of burst drug release and/or drug loading ii) and iii) a water-repelling backing film for unidirectional drug release, protection from wash-out by saliva and taste-masking.

FIG. 2A-2D show morphology of the multilayered DDS based on a peptide-loaded foam and drug-free mucoadhesive electrospun nanofibers—Nanofibers-on-foam-on-film (NFF) with 55.8±4.6 μg desmopressin loaded per patch. N=2-3, where N is the number of individual samples visualized.

FIG. 2A shows a cross-section of a multilayered DDS, where the foam/film and foam/fiber interfaces are enlarged. FIG. 2B shows the smooth surface of the peptide-loaded foam. FIG. 2C shows the rough surface of the peptide-loaded foam. FIG. 2D shows mucoadhesive electrospun chitosan/polyethylene oxide (PEO) nanofibers.

FIG. 3A-3D show mechanical properties of neat porous foam, foam with desmopressin (Foam Des), foam without plasticizers (Foam—P) and electrospun nanofibers. FIG. 3A show Stress-strain curve for the aforementioned samples. FIG. 3B shows Young's modulus. The Young's modulus was determined for the two distinct linear regions of the stress-strain curve for the nanofibers: Nanofibers-1 (strain from 0-0.4%) and Nanofibers-2 (0.6-1.0%). FIG. 3C shows ultimate tensile strength (UTS), and FIG. 3D shows elongation at break. N=2, n=5-8, where N is the number of individually produced batches and n is the number of samples analyzed per batch.

FIG. 4A-4B show that electrospun chitosan/PEO nanofibers improve mucoadhesion of biocompatible multi-layered DDS compared to foam. FIG. 4A shows the work of adhesion to ex vivo porcine buccal mucosa of: tape used for mounting the samples on the probe (control), foam with the rough and smooth surface facing the mucosa (foam possesses different topology after freeze-drying: one size has smooth surface, another side has rough surface), respectively, and nanofibers electrospun on either the rough or the smooth surface of the foam. N=3-7, where N is the number of repeats. Tissue samples obtained from at least two individual animals on two different days were included for each sample. *p<0.05.

FIG. 4B shows the evaluation of the biocompatibility of multi-layered NFF in vitro. The viability of human buccal TR146 cell monolayer after exposure to the foam, foam with backing film on the smooth surface (BS), multi-layered NFF and MiniRin® (60 μg of desmopressin, commercial drug product (orodispersible tablet)) relative to the control (cells exposed to HEPES-buffered Hanks Balanced Salt Solution (hHBSS), dashed line). Desmopressin (60 μg) was included as a control. N=2, n=3, where N is the number of cell passages and n is the number of samples tested per passage. The results are presented as mean±SD.

FIG. 5A-5C show the release of desmopressin from foam and from NFF multilayered DDS. FIG. 5A shows the release of desmopressin from either the smooth or the rough surface of the foam. No water-repelling backing layer was applied. Scanning electron microscopy (SEM) image of the smooth (SEM a1) and rough (SEM a2) surface of the foam. FIG. 5B shows the (significantly different, ***p<0.001) release of desmopressin from foam and multi-layered NFF with water-repelling film sprayed on the smooth surface (BS) of the foam (SEM b). FIG. 5C shows the release of desmopressin from foam and multi-layered NFF with water-repelling film sprayed on the rough surface (BR) of the foam (SEM c). N=5-9, where N is the number of repetitions from at least two batches. Results are presented as mean±SD.

FIG. 6 shows the permeation of desmopressin from multi-layered DDS and MiniRin® through ex vivo porcine buccal mucosa. The concentration of desmopressin in the receiver chamber was below the limit of quantification (LOQ were 6.8 ng/ml) of the method of quantification (liquid chromatography mass spectrometry) for all repetitions at all time points for mucosal tissue exposed to MiniRin® (240 μg desmopressin) in 2 mL isotonic buffer. The cumulative amount of permeated desmopressin from MinRin® tablets is therefore not displayed in the figure. N=6-7, where N is the number of repetitions (same batch of NFF but tissue from at least three different animals). Results are presented as mean±standard error of mean (SEM).

FIG. 7 shows representative scanning electron microscopy (SEM) images of the morphology of the smooth and rough surface of the drug-loaded foams (vitamin B2 (riboflavin), FITC-Dextran 4 kDa, FITC-Dextran 40 kDa or clotrimazole). N=1, where N represent the number of foam batches.

FIG. 8 shows the release profile of vitamin B2 (riboflavin), FITC-Dextran 4 kDa, FITC-Dextran 40 kDa or clotrimazole. The water-repelling backing layer was applied on the smooth surface of the foam for vitamin B2 (riboflavin), FITC-Dextran 4 kDa and FITC-Dextran 40 kDa, and release in hHBSS without BSA pH 6.8 was unidirectional. The release of clotrimazole was evaluated in hHBSS without BSA pH 6.8 with 33% (v/v) ethanol due to the low aqueous solubility of clotrimazole. No backing layer was applied on either side of the foam for release of clotrimazole, as ethanol would dissolve the water-repelling backing layer. N=1-2, n=2-4, where N represent the number of foam batches and n represents the number of repeats per batch. Results are presented as mean±standard deviation.

FIG. 9 shows the permeation of vitamin B2 (riboflavin) from NFF multi-layered DDS through ex vivo porcine buccal mucosa compared to vitamin B2 (riboflavin) in solution (50 μg/ml). N=3-4, where N represent the number of repeats (same batch of NFF but tissue was taken from three different animals). Results are presented as mean±standard error of mean (SEM).

### DETAILED DESCRIPTION OF THE INVENTION

Aspects of the present invention are described more fully hereafter with reference to the accompanying drawings.

The terminology used herein is for the purpose of describing particular aspects of the disclosure only, and is not intended to limit the disclosure. As used herein, the singular forms “a”, “an” and “the” are intended to include the plural forms as well, unless the context clearly indicates otherwise.

It should be noted that the word “comprising” does not necessarily exclude the presence of other elements or steps than those listed and the words “a” or “an” preceding an element do not exclude the presence of a plurality of such elements.

“Spinning” process means forming a fibrous product by the methods of e.g. electrospinning, wet spinning, dry spinning or melt spinning, and is here exemplified by fibers produced by the method of electrospinning.

“Dispersion” means any aqueous or organic liquid to which any polymer is added.

“Adhesive layers” means a layer that ensured a strong adhesion of the multilayered drug delivery system to the mucosa. The layer is composed of fibers.

“Water-repelling backing layer” means a layer promoting unidirectional release of the active pharmaceutical ingredient(s) towards the mucosa, thus preventing/delaying the release of the active pharmaceutical ingredient(s) in the opposite direction.

“Intermediate layer” means any layer in the multilayered drug delivery system positioned in between the adhesive layer and the water-repelling backing layer.

“Fibers” means elongated structures, typically in the form of a threadlike network prepared by methods of e.g. electrospinning, wet spinning, dry spinning or melt spinning.

Preparation processes comprise batch processing or continuous processing, in particular.

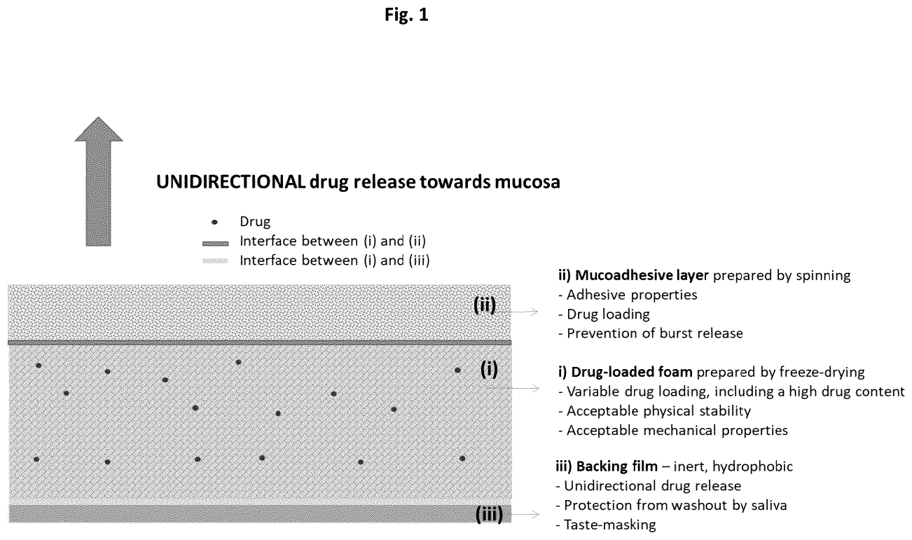

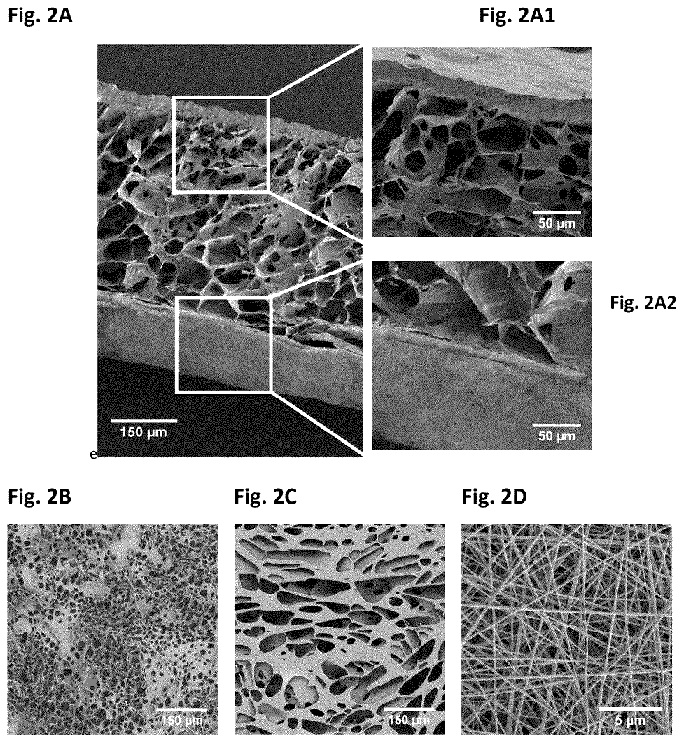

An embodiment of the present invention is shown e.g. in FIG. 1-2D including a dosage form for application on mucosa for release of at least one active pharmaceutical ingredient. The dosage form comprises an adhesive layer capable of adhering to the mucosa of the human or animal body, a water-repelling backing layer, and an intermediate layer positioned between the adhesive layer and the water-repelling backing layer. At least one active pharmaceutical ingredient may be incorporated in the intermediate layer and/or the adhesive layer. The intermediate layer may be a porous semi-solid or solid material, and the adhesive layer comprises fibres produced by the method of spinning. The dosage form may comprise two or more intermediate layers, each intermediate layer can comprise an active pharmaceutical ingredient, or may not comprise any active pharmaceutical ingredient.

Close connection between the individual layers of the invention provide a single dosage unit that can be handled, stored, marketed, distributed and administered as such.

The porous semi-solid or solid material of the intermediate layer may be prepared by freeze-drying or vacuum drying. The adhesive layer may comprise or consist of fibres produced by spinning.

The layers may comprise one or more hydrophilic and/or hydrophobic polymers and/or gums. The one or more hydrophilic and/or hydrophobic polymer and/or gums is/are selected from the group consisting of polyethylene oxide, methyl cellulose, hydroxypropyl cellulose, hydroxyethyl cellulose, carboxymethyl cellulose and its salts, methylhydroxyethylcellulose, hydroxypropyl methylcellulose, hydroxyethylcarboxymethylcellulose, carboxymethylcellulose, carboxymethylhydroxyethylcellulose, polyvinylpyrrolidone, chitosan, polyacrylic acid, polylactic acid, poly(lactic-co-glycolic acid), ethylcellulose, acacia, gellan gum, gelatin and polysaccharides, such as xanthan, agar, guar gum, carrageenan agar, alginic acid, polymethacrylate, and/or mixtures, and/or salts and/or derivatives hereof. Said one or more hydrophilic and/or hydrophobic polymer and/or gums may be in an aqueous or organic dispersion, or neat polymer, before lyophilisation (freeze-drying) and/or spinning and/or spraying.

The adhesive layer may comprise at least one adhesive polymer, comprising chitosan, alginate, polyethylene oxide and/or salt and/or derivatives hereof.

The intermediate layer may comprise of methyl cellulose, hydroxypropyl cellulose, hydroxyethyl cellulose, carboxymethyl cellulose, hydroxypropyl methylcellulose, xanthan, agar, guar gum, alginic acid, polymethacrylate, and/or mixtures, and/or salts and/or derivatives hereof.

The water-repelling backing layer may comprise of polyvinylpyrrolidone, ethylcellulose, polymethacrylates and/or mixtures and/or salt and/or derivatives hereof.

The active pharmaceutical ingredient may be selected from one or more of the groups consisting of small molecular entities, including lipids; and peptides, proteins, and oligonucleotides, whether prepared by synthesis or expression and/or extraction (biopharmaceuticals); and biological material, such as living cells; and particulate matter. The active pharmaceutical ingredient may comprise anti-inflammatory compounds, hormones, cannabinoids, opioids, immunosuppressive compounds, immune stimulating compounds e.g. vaccines, antibiotics, antivirals, antifungal, chemotherapeutics, probiotics, prebiotics, nutrients, vitamins, minerals, trace elements, and combinations hereof. The at least one active pharmaceutical ingredient may be present as dissolved, undissolved (droplets, amorphous or crystalline) or partly dissolved.

The porous semi-solid or solid material may be formed by freeze-drying or vacuum drying of an aqueous or organic polymer dispersion.

The adhesive layer comprises of fibers that may be formed by spinning, e.g., by electrospinning. The fibres may be produced from an aqueous polymer dispersion and/or an organic solvent or from neat polymer.

The intermediate layer and/or the adhesive layer may comprise at least one active pharmaceutical ingredient.

The covering of the intermediate layer with a water-repelling backing layer may be done by spraying or casting a polymer dispersion and/or by film formation by heating or light irradiation or by adhering a preformed water-repelling backing layer by wetting or heat. The covering of the intermediate layer with a water-repelling backing layer may be done by freeze-drying of the porous semi-solid or solid material onto the backing layer prepared by casting.

### EXAMPLES

### Example 1: Desmopressin, Proof-of-Principle

### Materials for Fabrication and Evaluation of Multi-Layered Drug Delivery System With Desmopressin

Chitoceuticals chitosan 95/100 (degree of deacetylation 96%, Mw 100-250 kDa, chitosan-96) was purchased from Heppe Medical Chitosan (Halle, Germany). Polyethylene oxide (Mw 900 kDa, PEO), bovine serum albumin (BSA), acetic acid anhydride, Hank's balanced salt solution (HBSS), Dulbecco's phosphate buffered saline (PBS), trifluoroacetic acid (TFA) and Dulbecco's modified Eagle's medium (DMEM), L-glutamine, penicillin, streptomycin, phenazine methosulfate (PMS), glycerol (≥99%), tributyl citrate, poly(ethylene glycol)-block-poly(propylene glycol)-block-poly(ethylene glycol) (Lutrol® F68), formic acid, and ethyl cellulose were obtained from Sigma Aldrich (St. Louis, MO, USA). Fetal bovine serum (FBS) was purchased from PAA laboratories (Brøndby, Denmark). 3-(4,5-dimethylthiazol-2-yl)-5-(3-carboxymethoxyphenyl)-2-(4-sulfophenyl)-2H-tetrazolium) (MTS) was obtained from Promega (Madison, WI, USA). N-2-hydroxyethylpiperazine-N′-2-ethanesulfonic acid (HEPES) was obtained from PanReac AppliChem (Damstadt, Germany). Acetonitrile was purchased from VWR (Søborg, Denmark). Polyethylene glycol 4000 (PEG 4000) and polyxyethylene sorbitan monolaurate (Tween® 20) was from Emprove Merck (Darmstadt, Germany). Iron(III)oxid (Secovit® E172) was from BASF (Copenhagen Denmark). Hydroxypropyl methylcellulose (HPMC) (Metolose® 60SH-4000) was kindly provided by Shin-Etsu (Chiyoda, Tokyo, Japan). The human TR146 cell line was obtained from European Collection of Authenticated Cell Cultures (ECACC) (Public Health England, Porton Down, UK) and purchased from Sigma Aldrich (St. Louis, MO, USA). Desmopressin as trifluoroacetic acid (TFA) salt (purity>98%) was obtained from SynPeptide (Shanghai, China). Ultrapure water (18.2 MΩ×cm) purified by a PURELAB flex 4 (ELGA High Wycombe, UK) was used if not otherwise mentioned.

### Freeze-Drying of Desmopressin-Loaded Porous Foam

The foam base was prepared according to Iftimi et al., (Iftimi et al., 2019, European Journal of Pharmaceutics and Biopharmaceutics, 136, 38-47) with slight modification in the composition of the formulation and manufacturing procedure. In short, 2.5000 g HPMC, 0.0825 poly(ethylene glycol)-poly(propylene glycol)-poly(ethylene glycol) (Lutrol® F68), 0.2500 g polyxyethylene sorbitan monolaurate (Tween® 20), 0.25 g PEG 4000 and 0.2500 g glycerol were dispersed in 50 mL ultrapure water preheated to 70° C. The mixture was stirred for 5 min and 50 mL ultrapure water (room temperature (RT) was added. This mixture was stirred on a magnetic stirrer until a clear viscous dispersion was obtained. The dispersion was stored at least overnight at 2-8° C. A total of 7.28 mg desmopressin-TFA (equal to 6 mg desmopressin) was added to 6.2 g of the prepared dispersion. For samples used for the ex vivo permeation study, 29.12 mg desmopressin-TFA (24 mg desmopressin) was added. Then, 5.1 g of the peptide-containing dispersion was cast in a glass Petri dish (area 66.6-71.8 cm²) and freeze-dried to yield the foam with a theoretical dose of 55±2 μg or 220±9 μg desmopressin per patch with a diameter of 10 mm. The freeze-drying was carried out on an Epsilon 2-4 LSC shelf apparatus (Martin Christ, Osterode am Harz, Germany). The casted formulation was cooled to −30° C. over 3 h and kept at this temperature for the next 3 h. After that, the pressure was reduced to 0.12 mbar over 10 min and the temperature was hereafter increased to 0° C. for 1 h 20 min. At this setting, the primary drying was conducted for 16.5 h. The obtained solid foams were removed from the Petri dish and stored in zipper bags over silica at 2-8° C. before use.

### Electrospinning a Mucoadhesive Layer of Nanofibers Onto Desmopressin-Loaded Foam

The mucoadhesive electrospun chitosan/PEO nanofibers were prepared by electrospinning according to Stie et al., (Stie et al., 2020, Carbohydrate Polymers, 116428) directly onto the freeze-dried foam. Briefly, a square of approximately 2 cm×2 cm was cut from the mat of freeze-dried foam and secured with adhesive tape on the aluminum foil on the stainless steel electrospinning collector. The nanofibers were electrospun from a 2% (w/w) chitosan with 0.7% (w/w) acetic acid and a 4% (w/w) PEO in ultrapure water and stirred for two days at RT. 30 min prior to electrospinning, the dispersions were mixed to obtain a 1:1 (w/w) ratio of the polymers in the dry fibers and hereafter electrospun for 2 h (20 kV, ES50P-10W high voltage source, Gemma High Voltage Research, Ormond Beach, Fl, USA) from a 20 G blunt needle (Photo-Advantage, Ancaster, ON, Canada) positioned 15 cm from the tip of the needle.

### Spraying a Repellant Backing-Layer On Foam and Nanofibers-On-Foam

A hydrophobic backing film was applied on either the rough or the smooth surface of the foam. 750 mg ethyl cellulose and 141 mg acetyl tributyl citrate and 47 mg glycerol as plasticizers were dispersed in 15 mL ethanol (absolute). After stirring for at least 3 h at RT, 10 mg iron(III)oxide pigment was added and the dispersion was hereafter stirred for at least 30 min. Discs (10 mm diameter) of the foam or fiber-on-foam were punched out using a biopsy puncher and the film was applied by spraying of the dispersion using an air brush (Model BD-134, Custom Colors, Jyderup, Denmark). The discs were kept in place during spraying on a plate with air-holes under vacuum.

### Evaluation of Morphology by Scanning Electron Microscopy (SEM)

The morphology of the foam, nanofibers, and multilayered DDS was visualized by SEM. The foam and the backing layer were visualized using a TM3030 SEM (Hitachi, Tokyo, Japan). For high-resolution SEM imaging of the electrospun nanofiber surface and cross-section of the multilayered DDS, the samples were visualized with a Quanta FEG 3D microscope (Thermo Fischer Scientific, Hillsboro, OR, USA). Prior to analysis, the samples were mounted on aluminum stubs on carbon tape and sputter-coated with gold (108 Auto sputter coater, Cressington Scientific Instruments, Watford, UK).

Evaluation of the Mechanical Properties of Foam and Multilayered DDS with Desmopressin

To prepare the mats for mechanical analysis, the chitosan-PEO dispersion was spun for 2 h, using the same process parameters as stated above. The electrospun mats and foams were stored in the desiccator over silica in the cold room (5-8° C.) and were let to equilibrate in the ambient conditions (21-24° C.) prior to analysis. The mechanical properties of the electrospun nanofibers, peptide-free and peptide-loaded foams were studied using a dynamic mechanical analyzer (DMA) (Q800, New Castle, DE, USA). The samples were prepared by cutting out rectangular shapes in dimension of 6.4 by 30.0 mm from the electrospun mats or freeze-dried foams. The cut out samples were measured for width and thickness at three different locations using a digital caliper, and the average values reported. The samples were mounted using film tension clamps. The preload force of 0.01 N and initial displacement of 0.01% were set up before actual analysis. The samples were subjected to a displacement ramp of 200 μm/min for a total length of 5000 μm. The obtained stress-strain curves were analyzed in Thermal Advantage Software v 5.5.2 (TA Instruments, New Castle, DE, USA) to determine Young's modulus as the slope of the curve in the initial linear region (0-1.0% strain for the foam samples, and 0-0.4% and 0.6-1.0% strain for nanofibers). Furthermore, the ultimate tensile strength (UTS) was determined as the maximum stress that the material could withstand before breaking, and the elongation at break was used to determine the strain at which the material could stretch anymore. The significance of the results was evaluated using unpaired Student's t-test with equal variances (confirmed by F-test).

### Evaluation of the Mucoadhesion of Foam and Nanofibers-On-Foam With Desmopressin

The mucoadhesion of the foam and the fiber-on-foam multilayered system to ex vivo porcine buccal mucosa was evaluated according to Stie et al., (Stie et al., 2020, Carbohydrate Polymers, 116428) with small modifications. In short, cheeks from healthy experimental pigs (approximately 30-60 kg, Danish Landrace/Yorkshire/Duroc) were collected immediately after euthanization and kept in PBS on ice until use on the same day, as the tissue was isolated. The cheeks were trimmed to remove the underlying tissue and cut to a thickness of 0.25-0.50 mm with an electric dermatome (Zimmer Biomet, Albertslund, Denmark). The buccal epithelium was immediately mounted on microscopy glass slides using Loctite® Power Flex gel (Henkel, Ballerup, Denmark) and kept submerged in PBS on ice until use; measurements were conducted on the same day as tissue isolation. The force of adhesion of discs (10 mm in diameter) to ex vivo porcine buccal mucosa was determined at RT by a TA.XT plus texture analyser (Stable Micro Systems, Godalming, UK) employed with a 5 kg load cell. The samples were in contact with the buccal tissue for 10 s by applying a force of 500 g, and withdrawn with a speed of 10 mm/s. The work of adhesion was determined as the area under the recorded force versus distance curve using the Exponent software (Stable Micro Systems, Godalming, UK).

### Release of Desmopressin From Foam and Multilayered DDS

Discs (10 mm in diameter) of foam and fiber-on-foam with and without water-repelling backing film were fixed in Ussing chamber sliders (diffusion area of 0.4 cm²) and placed in EM-CSY-8 Ussing chambers (Physiologic Instruments, Santiago, CA, USA). 2 mL warm (37° C.) 10 mM HEPES in HBSS pH 6.8 with 0.05% (w/v) BSA (hereafter hHBSS) was added to each side of the sample. The samples were incubated for 3 h at 37° C. and aliquots of 100 μL were withdrawn from each of the diffusion cells at specific time points and replenished with 100 μL warm (37° C.) hHBSS. The exact peptide dose was determined by disintegrating a 10 mm foam disc of known weight in 1 mL water for at least 1 h at RT. All samples were centrifuged (10,000 rpm/9279×g, 10 min, 4° C.) and the concentration of desmopressin in the supernatant was determined by reversed phase high performance liquid chromatography with UV detection (RP-HPLC-UV).

### Quantification of Ddesmopressin by RP-HPLC-UV

The analysis was conducted on a Shimadzu Prominence system (Kyoto, Japan) with a Kinetex XB-C18 column (100×2.1 mm, 3.6 μm, Phenomenex, Torrance, CA, USA). Desmopressin was eluted using a mobile phase consisting of eluent A [95:5% (v/v) acetonitrile:water, 0.1% (v/v) TFA] and eluent B [5:95% (v/v) acetonitrile:water, 0.1% (v/v) TFA]. Samples were run with a gradient of 0→40% eluent B over 8 min at 0.8 mL/min at 40° C. The limit of detection (LOD) and limit of quantification (LOQ) were 0.6 μg/mL and 1.7 μg/mL respectively. The significance of the results was evaluated using a multiple unpaired Student's t-test for each time point. Test of for equal variances was performed for each time point (F-test).

### In Vitro Compatibility Testing of Foam and Multilayered DDS With Desmopressin

TR146 cells were cultured in DMEM supplemented with FBS (10% (v/v)), L-glutamine (2 mM), penicillin (100 U/mL) and streptomycin (100 μg/mL) in Corning Costar® polystyrene culture flasks (175 cm², Sigma Aldrich, St. Louis, MO, USA) 37° C. with 5% CO₂ in an humidified environment. A total of 85,000 TR146 cells/well were seeded in flat-bottom, transparent 12-well Nunclon™ delta cell culture-treated plates (3.5 cm², Thermo Scientific, Roskilde, Denmark) and cultured for three days at the aforementioned conditions attaining a confluency of 70-90% before use. The cells were washed twice in 2 mL 37° C. hHBSS without BSA. The cells were exposed to desmopressin, foam, foam with backing film, the multilayered DDS system, or a MiniRin® (60 μg desmopressin) freeze-dried sublingual tablet submerged in 2 mL hHBSS and incubated for 3 h at 37° C. with mild agitation (50 rpm). After exposure, remnants of the formulations were removed and the cells were washed twice with 2 mL warm (37° C.) hHBSS without BSA. The cells were then incubated at 37° C. for up to 2 h with 1 mL solution containing 240 μg/mL MTS and 2.4 mg/mL PMS in hHBSS without BSA. Subsequently, 100 μl samples in quadruplicate of the solution with metabolized MTS were transferred from each well to a transparent 96-well plate and the absorbance at 492 nm was measured in a plate reader (POLARstar OPTIMA, BMG LABTECH, Ortenberg, Germany). The absorbance of the unreacted MTS/PMS solution was defined as the blank (Abs_{blank}, 0% cell viability), while the control was defined as cells incubated with hHBSS (Abs_{control}, 100% cell viability). The relative cell viability was determined (Eq. 2):

$$Relative cell viability ( \% ) = \frac{{Abs}_{sample} - {Abs}_{blank}}{{Abs}_{control} - {Abs}_{blank}} \cdot 100 \% \qquad Eq . 2$$

The osmolality was determined on an Osmomat 3000 Freezing point osmometer (Genotec, Berlin, Germany) and the pH by a SenTix MIC pH electrode (VWR, Soeborg, Denmark).

### Permeation of Desmopressin Through Ex Vivo Porcine Buccal Mucosa

Cheeks from healthy experimental pigs (approximately 30-60 kg, Danish Landrace/Yorkshire/Duroc) were collected immediately after euthanization and kept in PBS on ice until use on the same day as harvesting the tissue. The cheeks were trimmed to remove the underlying tissue and cut to a thickness of 0.75 mm with an electric dermatome (Zimmer Biomet, Albertslund, Denmark) and mounted in Ussing sliders (diffusion area of 0.4 cm²) and placed in EM-CSY-8 Ussing chambers (Physiologic Instruments, Santiago, CA, USA). NFF was placed on the buccal epithelium and mounted in the Ussing sliders. A layer of parafilm was applied to ensure contact between the NFF patches and the tissue. As a control, tissue were exposed to 2×MiniRin® (120 μg desmopressin/tablet) tablets in 2 mL hHBSS. The receiver chamber contained hHBSS (adjusted to pH 7.4). Aliquots of 100 μL were withdrawn from the receiver chamber over a 5 h period at 37° C. and replaced with warm (37° C.) hHBSS (adjusted to pH 7.4).

### Quantification of Desmopressin by High Performance Liquid Chromatography Mass Apectrometry (LC-MS)

100 μL samples were mixed with 100 μL precipitation buffer (prepared by dissolving 2 g ZnSO₄·7H₂O in 55 mL purified water and 50 mL acetonitrile) and centrifuged (20,000×g, 10 min, RT). The supernatant was analyzed by LC-MS on a Thermo Accela HPLC system coupled to a Thermo TSQ Vantage triple quadrupole mass spectrometer (Thermo Fisher Scientific, San Jose, CA, USA). The injection volume was 30 μL on a Kinetex XB-C18 column (50 mm×2.1 mm, 2.6 μm) (Phenomenex, Torrance, CA, USA). Desmopressin was eluted using a mobile phase consisting of eluent A [0.1% (v/v) formic acid in ultrapure water] and eluent B [0.1% (v/v) formic acid in acetonitrile]. Samples were run with a gradient of 5%→28% eluent B over 5 min at 0.8 mL/min at 40° C. Samples were analyzed in single reaction monitoring (SRM) mode with electro-spray ionization in positive ion mode detecting desmopressin by monitoring the transition pairs m/z 535.37 precursor ion to m/z 328.4 product ion. The limit of detection LOD and LOQ were 2.3 ng/ml and 6.8 ng/ml, respectively. The data was processed using Skyline 20.1.0.155 (MacCoss Lab, Department of Genome Science, University of Washington, Seattle, WA, USA). For calculation of the average cumulative permeation across ex vivo porcine mucosa of desmopressin released from NFF, samples with a concentration of desmopressin below the LOQ were set to LOQ/2 i.e. 3.4 ng/ml.

### Examples 2-5: Vitamin B2 (riboflavin), FITC-Dextran 4 kDa, FITC-Dextran 40 kDa and Clotrimazole

Materials for Fabrication and Evaluation of Multi-Layered Drug Delivery System With Vitamin B2 (Riboflavin), FITC-Dextran 4 kDa, FITC-Dextran 40 kDa and Clotrimazole

Chitoceuticals chitosan 95/100 (degree of deacetylation 96%, Mw 100-250 kDa, chitosan-96) was purchased from Heppe Medical Chitosan (Halle, Germany). Polyethylene oxide (Mw 900 kDa, PEO), bovine serum albumin (BSA), acetic acid anhydride, Hank's balanced salt solution (HBSS), Dulbecco's phosphate buffered saline (PBS), glycerol (≥99%), tributyl citrate, poly(ethylene glycol)-block-poly(propylene glycol)-block-poly(ethylene glycol) (Lutrol® F68), ethyl cellulose, FITC-Dextran 4 kDa and FITC-Dextran 40 kDa were obtained from Sigma Aldrich (St. Louis, MO, USA). Vitamin B2 (riboflavin, Ph. Eur. 4^{th} Ed.) was from Unikem (Copenhagen, Denmark) and clotrimazole (Ph. Eur) was from Fagron (Uitgeest, The Netherlands). Polyethylene glycol 4000 (PEG 4000) and polyxyethylene sorbitan monolaurate (Tween® 20) was from Emprove Merck (Darmstadt, Germany). Iron(III)oxid (Secovit® E172) was from BASF (Copenhagen Denmark). Hydroxypropyl methylcellulose (HPMC) (Metolose® 60SH-4000) was kindly provided by Shin-Etsu (Chiyoda, Tokyo, Japan). Hydrochloric acid (5 M), methanol and ethanol (absolute) were purchased from VWR (Søborg, Denmark). Ultrapure water (18.2 MΩ×cm) purified by a PURELAB flex 4 (ELGA High Wycombe, UK) was used if not otherwise mentioned.

### Freeze-Drying of Drug-Loaded (Examples 2-5) Porous Foam

The foam base was prepared according to Iftimi et al., (Iftimi et al., 2019, European Journal of Pharmaceutics and Biopharmaceutics, 136, 38-47) with slight modification in the composition of the formulation and manufacturing procedure. In short, 2.5000 g HPMC, 0.0825 poly(ethylene glycol)-poly(propylene glycol)-poly(ethylene glycol) (Lutrol® F68), 0.2500 g polyxyethylene sorbitan monolaurate (Tween® 20), 0.25 g PEG 4000 and 0.2500 g glycerol were dispersed in 50 mL ultrapure water preheated to 70 ° C. The mixture was stirred for 5 min and 50 mL ultrapure water (room temperature (RT) was added. This mixture was stirred on a magnetic stirrer until a clear viscous dispersion was obtained. The dispersion was stored at least overnight at 2-8° C. 4.42 g of the prepared dispersion was added to 3.69 mg, 0.735 mg, 0.735 mg or 2.21 mg for vitamin B2 (riboflavin), FITC-Dextran 4 kDa, FITC-Dextran 40 kDa or clotrimazole, respectively. For loading of clotrimazole, 10 μl 1 M HCl was also added. Then, 1.47 g of the drug-containing dispersion was cast in a glass Petri dish (area 9.6 cm²) and freeze-dried to yield the foam with a theoretical dose of 25.23 μg, 5.13 μg, 5.13 μg or 15.27 μg per mg foam for vitamin B2 (riboflavin), FITC-Dextran 4 kDa, FITC-Dextran 40 kDa or clotrimazole, respectively. The freeze-drying was carried out on an Epsilon 2-4 LSC shelf apparatus (Martin Christ, Osterode am Harz, Germany). The casted formulation was cooled to −30° C. over 3 h and kept at this temperature for the next 3 h. After that, the pressure was reduced to 0.12 mbar over min and the temperature was hereafter increased to 0° C. for 1 h 20 min. At this setting, the primary drying was conducted for 16.5 h. The obtained solid foams were removed from the Petri dish and 10 stored in zipper bags over silica at 2-8° C.before use.

### Electrospinning a Mucoadhesive Layer of Nanofibers Onto Drug-Loaded Foam (Examples 2-5)

The mucoadhesive electrospun chitosan/PEO nanofibers were prepared by electrospinning according to Stie et al., (Stie et al., 2020, Carbohydrate Polymers, 116428) directly onto the freeze-dried foam. Briefly, the freeze-dried foam was removed from the Petri dish and the edges of the foam were cut, and the foam was secured with adhesive tape on the aluminum foil on the stainless steel electrospinning collector. The nanofibers were electrospun from a 2% (w/w) chitosan with 0.7% (w/w) acetic acid and a 4% (w/w) PEO in ultrapure water that were beforehand stirred for two days at RT. 30-60 min prior to electrospinning, the dispersions were mixed to obtain a 2:3 (w/w) ratio of chitosan to PEO in the dry fibers and hereafter electrospun (Linari Engineering S.R.L., Pisa, Italy) for 3 h from a 20 G blunt needle (Photo-Advantage, Ancaster, ON, Canada) positioned 15 cm from the tip of the needle at a relative humidity (RH)<10% and a temperature of 23-26° C.

### Spraying a Repellant Backing-Layer On Foam and Nanofibers-On-Foam

A water-repelling backing film was applied in the same manner as previously described under example 1.

### Evaluation of Morphology by Scanning Electron Microscopy (SEM)

The morphology of the foam, nanofibers, and multilayered DDS was visualized by SEM. The foam and the backing layer were visualized using a TM3030 SEM (Hitachi, Tokyo, Japan). Prior to analysis, the samples were mounted on aluminum stubs on carbon tape and sputter-coated with gold (108 Auto sputter coater, Cressington Scientific Instruments, Watford, UK).

Release of Vitamin B2 (Riboflavin), FITC-Dextran 4 kDa and FITC-Dextran 40 kDa from NFF Multi-Layered DDS

Discs (10 mm in diameter) of NFF multilayered DDS were fixed in Ussing chamber sliders (diffusion area of 0.4 cm²) and placed in EM-CSY-8 Ussing chambers (Physiologic Instruments, Santiago, CA, USA). 2 mL warm (37° C.) hHBSS without BSA pH 6.8 was added to each side of the sample. The samples were incubated for 3 h at 37° C. and aliquots of 100 μL were withdrawn from each of the diffusion cells at specific time points and replenished with 100 μl warm (37° C.) hHBSS without BSA pH 6.8. The loading efficiency was determined by disintegrating 10 mm foam discs of known weight in 2 mL hHBSS without BSA pH 6.8 for at least 2 h at RT.

### Permeation of Vitamin B2 (Riboflavin) Through Ex Vivo Porcine Buccal Mucosa

Cheeks from healthy experimental pigs (approximately 30 kg, Danish Landrace/Yorkshire/Duroc) were collected immediately after euthanization and kept in PBS on ice until use on the same day as harvesting the tissue. The cheeks were trimmed to remove the underlying tissue and cut to a thickness of 0.75 mm with an electric dermatome (Zimmer Biomet, Albertslund, Denmark) and mounted in Ussing sliders (diffusion area of 0.4 cm²) and placed in EM-CSY-8 Ussing chambers (Physiologic Instruments, Santiago, CA, USA). NFF was placed on the buccal epithelium and mounted in the Ussing sliders. A layer of parafilm was applied to ensure contact between the NFF patches and the tissue. As a control, tissues were exposed to 2 ml of 50 μg/mL vitamin B2 (riboflavin) in hHBSS pH 6.8. The receiver chamber contained hHBSS adjusted to pH 7.4. Aliquots of 100 μL were withdrawn from the receiver chamber over a 5 h period at 37° C. and replaced with warm (37° C.) hHBSS adjusted to pH 7.4.

### Quantification of Vitamin B2 (Riboflavin), FITC-Dextran 4 kDa and FITC-Dextran 40 kDa

Samples (100 μl) for vitamin B2 (riboflavin), FITC-Dextran 4 kDa and FITC-Dextran 40 kDa collected from the release experiment or permeation study (for vitamin B2 (riboflavin) were directly pipetted into a black flat-bottomed transparent 96-well plate and the fluorescence was recorded on a platereader (POLARstar Omega, BMG LABTECH, Ortenberg, Germany) at an excitation wavelength of 485 nm and emission wavelength of 520 nm.

Release of Clotrimazole from Multi-Layered DDS

Discs (10 mm in diameter) of nanofibers on foam (no water-repelling backing-layer) were placed in 2 mL warm (37° C.) in hHBSS without BSA pH 6.8 with 33% (v/v) ethanol (absolute) in a 12 well plate. The samples were incubated for 3 h at 37° C. and aliquots of 100 μL were withdrawn at specific time points and replenished with 100 μL warm (37° C.) hHBSS without BSA pH 6.8 with 33% (v/v) ethanol (absolute). The loading efficiency was determined by disintegrating 10 mm foam discs of known weight in 2 mL hHBSS without BSA pH 6.8 with 33% (v/v) ethanol (absolute) for at least 2 h at RT.

### Quantification of Clotrimazole by RP-HPLC-UV

All samples were centrifuged (10,000 rpm/9279×g, 10 min, 4° C.) and the concentration in the supernatant was determined. The analysis was conducted on a Shimadzu Prominence system (Kyoto, Japan) with a Kinetex EVO-C18 100 Å column (150 mm×4.6 mm, 5 μm, Phenomenex, Torrance, CA, USA). Clotrimazole was eluted by isocratic separation using a mobile phase consisting of 80:20% (v/v) methanol:water at 1.5 mL/min at 40° C. and detected at a wavelength of 229 nm.

### Results

### Example 1: Characterization of Multi-Layered Drug Delivery System and Proof-of-Principle by Loading of the Peptide Desmopressin in NFF

### Mucosadhesive Peptide-Loaded Multilayered DDS for Unidirectional Release

The new DDS was explored for its ability to enhance the permeation of a drug across the (oral) mucosa by retaining a high concentration of drug at the site of application and ensure unidirectional drug release towards the mucosa for a prolonged period of time. The multilayered NFF technology comprises three layers: i) mucoadhesive electrospun nanofibers, ii) a peptide-loaded foam, and iii) a water-repelling backing film (FIG. 1). Each of the layers of the NFF served a specific purpose and different methods were applied to achieve optimal properties of the three layers. The peptide-loaded foam was prepared by freeze-drying and served as a reservoir of the active pharmaceutical ingredient/drug. The peptide desmopressin was encapsulated in the foam as a proof-of-principle and the dose was 55.8±4.6 μg (N=5, n=3-4, where N is the number of individual batches and n is the number of samples per batch) desmopressin per dosage form of NFF (discs of 10 mm in diameter). The specific loading of desmopressin was 28.2±0.2 μg per mg of foam.

The peptide-loaded freeze-dried foam showed a two-sided morphology; a smooth surface with small and uniformly distributed pores (oriented towards the Petri dish during freeze-drying) (FIG. 2B), and a rough surface with larger pores (from this side ice crystals were sublimated) (FIG. 2C). Mucoadhesive chitosan/PEO nanofibers were electrospun on the surface of the foam to ensure efficient adhesion of the multi-layered DDS to the mucosa (FIG. 2D). The chitosan/PEO nanofibers were electrospun in water with minimum amounts of acetic acid (0.7% (w/w)) as a solvent. The electrospun nanofibers were uniform without artifacts and had a mean diameter of 167±27 nm (N=3, n=100) as previously described (Stie et al., 2020, Carbohydrate Polymers, 116428). A thin water-repelling backing film based on the hydrophobic polymer ethyl cellulose was applied to the porous foam to ensure unidirectional peptide release and protect the DDS from peptide washout by saliva upon prolonged adhesion of the DDS to the oral mucosa (FIG. 2A). Cross-sections of the NFF multi-layered system clearly showed a tight and even connection between the distinctive layers of the film, the porous foam and the electrospun nanofibers (FIG. 2A). From a technical point of view, it is worth noting that the multi-layered system demonstrates the possibility of electrospinning a separate layer of mucoadhesive nanofibers on a solid substrate; here the foam. Importantly, all processes only use non-hazardous and environmentally friendly solvents/dispersion media.

Drugs can be encapsulated in nanofibers by co-electrospinning the drug with the polymer blend. Although electrospinning is a very versatile technique, some drugs or excipients may have limited spinability. Surfactants and organic solvents have often been used by others to improve the electrospinability of dispersions by lowing the surface tension of the dispersion and to enhance evaporation of the solvent during spinning. However, the use of such potentially harsh conditions compromises the safety of the DDS and might furthermore reduce the stability of the drug to be encapsulated, why the NFF technology use another approach; combination with foams. As demonstrated, freeze-drying is a supplementary technique to electrospinning for the production of dry drug-loaded patches. Incorporation of the drug can be done in-process, but the foam is also suitable for loading of drugs by absorption post preparation (Iftimi et al., 2019, European Journal of Pharmaceutics and Biopharmaceutics, 136, 38-47). The presented NFF multi-layered DDS provides an opportunity for loading of a variety of drugs or excipients in the foam and/or in the electrospun nanofibers either by in-process or post-process incorporation. The drug loading can reach at least 12 mg per 1 cm² of the foam (Edinger et al., 2019, AAPS PharmSciTech 20, 207). At least two drugs were possible to incorporate into the foam.

### Mechanical Properties of Foam and Nanofibers

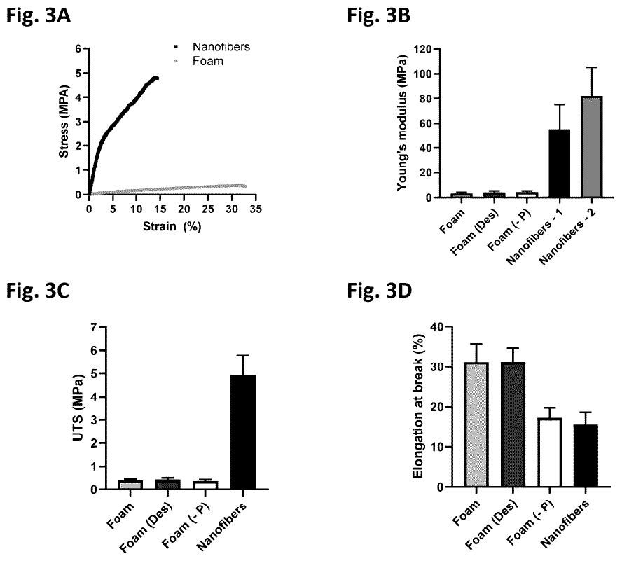

The optimal mechanical properties of the drug delivery systems are crucial to allow for robust processing, transportation and for overall handling feasibility of the dosage form such as ease in taking out the dosage form from the package and application to the site of drug absorption by a patient. Furthermore, the NFF patch needs to be flexible to allow close adhesion to the curved surfaces of body. In light of that, the mechanical characteristics of the foam and nanofibers were studied in the tension mode. Both foam and nanofibers showed behavior typical for ductile material (FIG. 3A). Importantly, both nanofibers, foam and film are very bendable both alone and when combined, and thus can be handled without breaking. The stress-strain curves of nanofibers consisting of PEO and chitosan (1:1 (w/w)) possessed a low slope linear region, followed by a linear region with a stiffer slope in the very beginning of the curve (FIG. 3B). The foam appeared to possess superior flexibility as compared to nanofibers that were stiffer (FIG. 3B). Inclusion of peptide as a drug in the foam did not have a significant effect on the rigidity of the sample as the samples displayed statistically insignificant (p>0.05) different Young's modulus values. None of the samples showed a well-defined yield point, which indicates the limit of elastic behavior and the beginning of plastic behavior. Surprisingly, nanofibers appeared to be much stronger than the foam samples (FIG. 3C). The later had, however, superior ability to stretch, when the foam formulation contained plasticizers (FIG. 3D). The inclusion of desmopressin (55 μg/dose) in general did not affect the mechanical properties of the foam.

### Physical Stability of the NFF

The drug-free foam is stable in ambient conditions for over 4 years. No morphological differences were observed in the foam after storing it in different levels of relative humidity (0-90%) for at least 3 months (Iftimi et al., 2019, European Journal of Pharmaceutics and Biopharmaceutics, 136, 38-47). Temperature did not also have any effect on the morphology of drug-free foam (Öblom et al., International Journal of Pharmaceutics, 589, 119866). Desmopressin-loaded foam had a physical stability for at least a year after storing at 2-8° C. over silica.

### Strong Adhesion of Multi-Layered DDS to Porcine Buccal Mucosa Ex Vivo

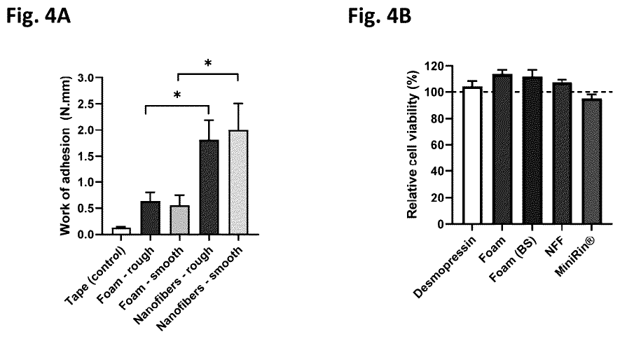

Mucoadhesion is an important property to ensure close contact between the DDS and the mucosa to retain a high concentration of drug at the site of absorption, thereby enhancing total drug diffusion across the mucosal barrier to reach the systemic circulation. The mucoadhesive properties of the nanofiber-on-foam DDS were evaluated by measuring the work of adhesion to ex vivo porcine buccal mucosa. The foam alone had limited adhesion to the ex vivo porcine buccal mucosa (FIG. 4A) with no difference found between the more (rough) and less (smooth) porous surface of the drug-loaded foam (p>0.05). The presence of a layer of electrospun chitosan/PEO nanofibers on the foam significantly (p<0.05) improved the mucoadhesive properties of the multi-layered DDS. Indeed, the work of adhesion was more than three times higher for the fiber-on-foam multi-layered DDS compared to the adhesion of the foam alone. By visual inspection, the fiber-on-foam multi-layered DDS appeared to swell and the underling tissue was dehydrated after detachment of the DDS from the buccal tissue, which indicates that the adhesion of the DDS to the mucosa was driven by the hygroscopic nature of the chitosan/PEO nanofibers. This was supported by that the fibers did not separate from the foam in the mucoadhesion test. For reasons of comparison, an evaluation of the adhesion of MiniRin® freeze-dried tablets to ex vivo porcine buccal mucosa was attempted, but the marketed tablets disintegrated instantaneously in the presence of the wetted tissue and the measurement could not be conducted.

Only biocompatible excipients were included in the formulation of the foam, the electrospun nanofibers, and the backing film. To demonstrate biocompatibility of the proposed multilayered DDS, the biocompatibility of the fiber-on-foam multi-layered DDS was evaluated in vitro by exposing monolayers of human buccal TR146 cells to NFF multi-layered DDS discs of 10 mm in diameter, their individual components i.e. the foam with or without backing and content of desmopressin, or marketed MiniRin® freeze-dried tablets. No changes in physiological buffer pH and osmolality compared to the control were recorded in the presence of the fiber-on-foam multi-layered DDS, whereas a slight increase in apical buffer osmolality from 300 mOsmol/kg to 337±10 mOsmol/kg was observed for cells treated with MiniRin®. All samples tested were equivalent to one dose of 55.8±4.6 μg desmopressin. As expected, none of the evaluated samples affected the viability of the buccal TR146 cells significantly compared to the control (FIG. 4B).

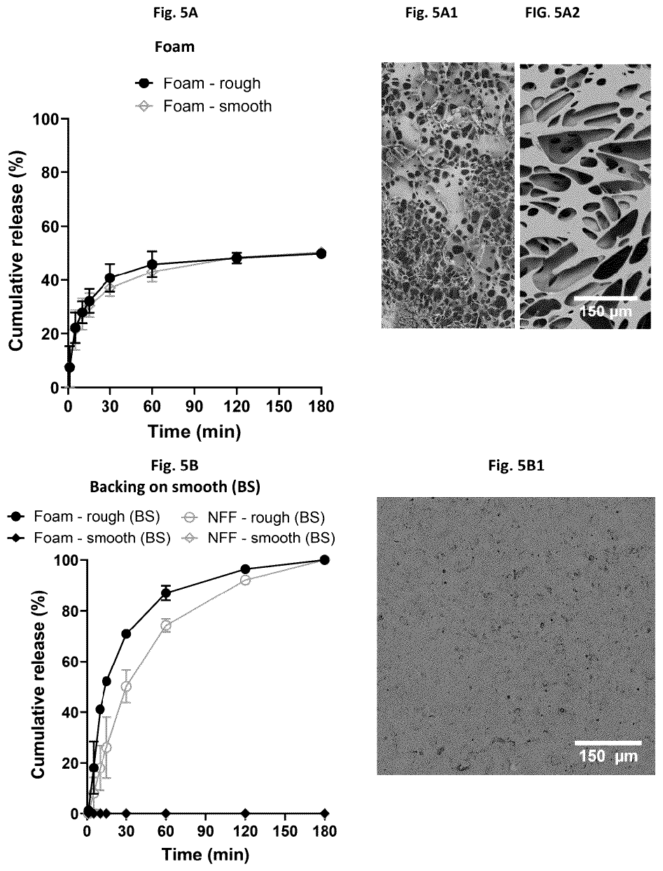

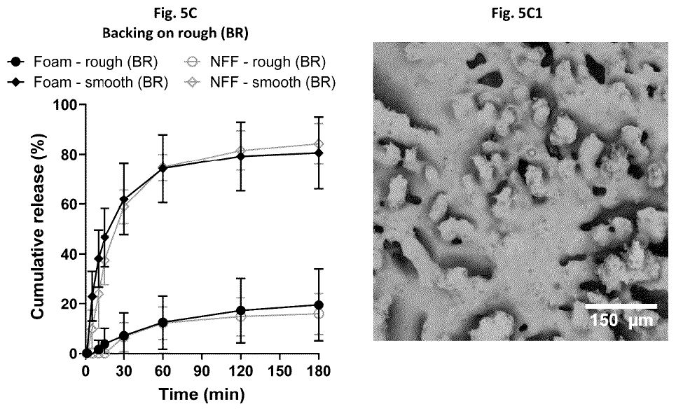

Controlled and unidirectional release of desmopressin from multi-layered fiber-on-foam DDS Controlled and unidirectional release is crucial to limit the loss of drug by the salivary flow and to ensure a high concentration gradient of drug across the mucosa for a prolonged period of time. A continuous film with full coverage of the small pores in the foam was achieved after application of the hydrophobic water-repelling film matrix on the smooth surface of the foam (FIG. 5B). In contrast, the larger pores in the foam were still visible by SEM after application of the backing layer to the rough surface of the foam, which indicates incomplete coverage of the pores on the surface of the foam (FIG. 5C).

The release of desmopressin from the NFF multi-layered DDS was evaluated. The DDS was placed between two diffusion chambers, and the release of peptide into each of the chambers was determined over time. The foam disintegrates very fast in the aqueous test medium leading to rapid drug release to both chambers. In the absence of electrospun nanofibers and a water-repelling backing layer, around 80% of the total amount of desmopressin was released from the foam after 30 min, and with an equal amount of desmopressin (around 40%) released from each of the two surfaces of the foam (rough and smooth) (FIG. 5A). In contrast, the layer of electrospun chitosan/PEO nanofibers and water-repelling backing film were still intact after 3 h in physiological buffer. Unidirectional release of desmopressin was accomplished with the spraying of the water-repelling film on the smooth surface of the foam (FIG. 5B). In contrast, unidirectional release was not fully achieved with application of the backing layer on the rough surface of the foam as about 20% of the total amount of released desmopressin was detected in the diffusion chamber fronting the backing layer after 3 h (FIG. 5C). This is in good correlation with the SEM images, which showed insufficient coverage of the bigger pores and full coverage of the smaller pores of the rough and smooth surface of the foam, respectively. Furthermore, electrospun nanofibers on the rough surface of the foam significantly (p<0.001) decreased the rate of desmopressin release (FIG. 5B). This indicates that the mucoadhesive electrospun chitosan/PEO nanofibers constitute a thin diffusion barrier for wetting of the drug-loaded foam and thus decrease the release rate of the drug.

### NFF Multi-Layered DDS Improves Permeation of Desmopressin Across Buccal Mucosa Ex Vivo

In general, mucosal membranes constitute a great permeation barrier for drug delivery. It was demonstrated that close adhesion of the NFF drug delivery system to the ex vivo porcine buccal mucosa increased the amount of permeated peptide (to 203±14 μg/dose or 259±14 μg/cm², N=4, where N is the number of samples) compared to desmopressin from MiniRin® tablets (240 μg) dissolved in 2 mL physiological buffer (hHBSS). The permeated amount of desmopressin from commercial MiniRin® tablets was below the limit of quantification (LOQ) with the used quantification method (LC-MS) for all repeats at all time points (FIG. 6). In contrast, the permeation of desmopressin from NFF after just one h was on average higher than the LOQ for the method of quantification (LC-MS) and thus higher than the permeation of desmopressin from MiniRin® tablets. This indicates that the NFF system indeed have the potential to improve the delivery of drugs across the oral mucosa compared to marketed formulations for oromucosal delivery, e.g. orodispersible tablets or films.

### Examples 2-5: Demonstration of the Versatility of Multilayered NFF Drug Delivery System

### Encapsulation and Controlled Release of Drugs/Molecules of Various Molecular Weights and Hydrophobicity/Hydrophilicity in NFF

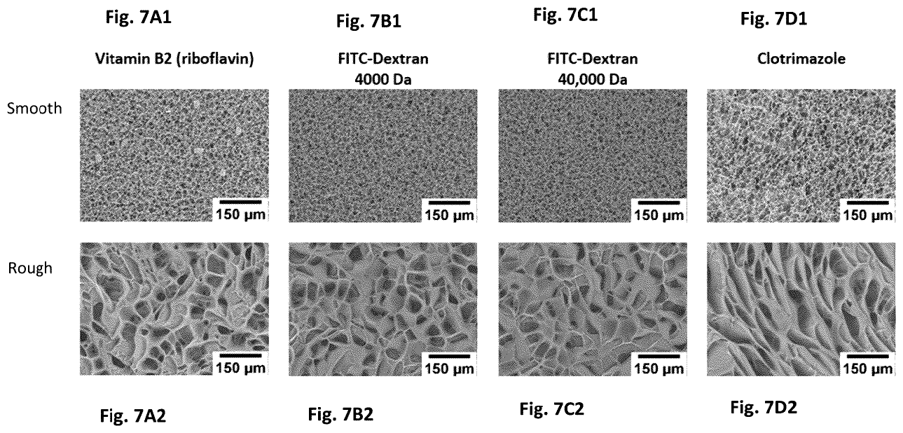

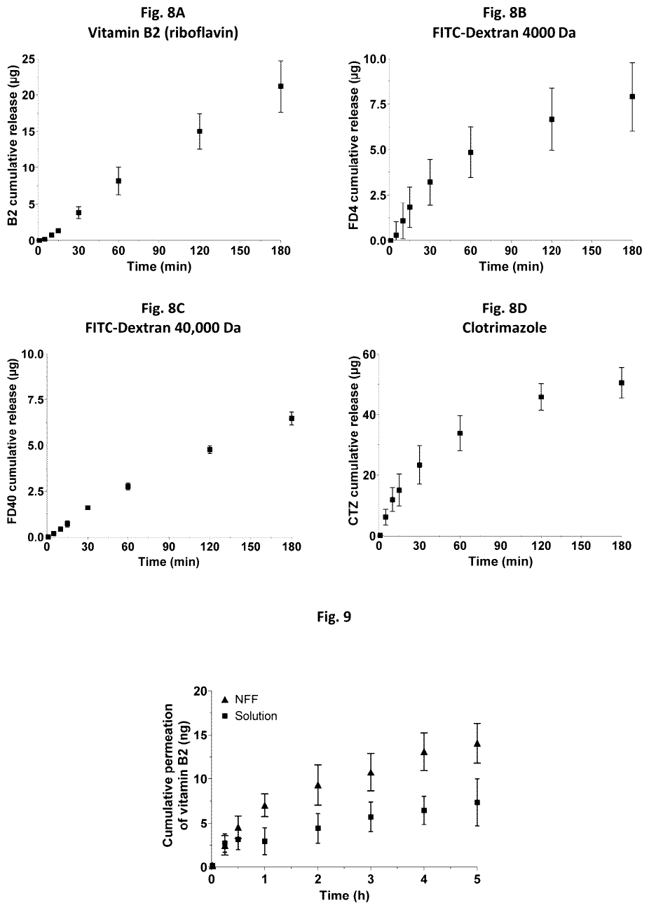

One of the greatest benefits of the multilayered NFF drug delivery system over existing patch technologies made, e.g., solely from electrospun nanofibers is the great versatility in terms of drug types that can be loaded. To demonstrate the great versatility of the NFF multilayered drug delivery system, drugs/molecules of various molecular weights and hydrophilicity/hydrophobicity (Table 1, below) were loaded in the NFF (FIG. 7) and their release was evaluated (FIG. 8). Of the hydrophilic drugs/molecules included in this evaluation, vitamin B2 (riboflavin) has a molecular weight of 376.4 Da, desmopressin is a peptide of 1069.2 Da (aforementioned) and FITC-Dextran of 4000 Da and 40.000 Da are fluorescent-labelled glycans that is often used as models for large macromolecules such as proteins. Clotrimazole is a small and hydrophobic molecule drug with a very low aqueous solubility (Log P 6.3, 0.49 mg/L in water; from Huang et al., 2020, International Journal of Pharmaceutics, 119811) that is used for the treatment of fungal infection e.g. candidiasis in the mouth and thus of relevance for oromucosal delivery by a system such as NFF. The minimal inhibitory concentration (MIC) and minimal fungicidal concentration (MFC) of clotrimazole to C. albicans that can be responsible for oromucosal candidiasis has been determined to be 1 and 4 μg/mL, respectively (Huang et al., 2020, International Journal of Pharmaceutics, 119811) and hence relevant doses (15.27 μg clotrimazole/mg foam) could be loaded in NFF. The drugs were all successfully loaded in the porous foam of the NFF, and as visualized by SEM, the foam showed the characteristic two-sided morphology (FIG. 7). The loading was determined for each of the drugs (Table 1, below) and controlled release was confirmed (FIG. 8). Crystals were visible on the smooth side of foam loaded with vitamin B2 (riboflavin) as visualized by SEM., which could indicate that vitamin B2 was undissolved or that the vitamin B2 (riboflavin) has recrystallized in the foam (FIG. 7). Thus, encapsulation of drugs/molecules of both low and high molecular weight, hydrophilic/hydrophobic drugs and as dissolved and/or undissolved were indeed demonstrated. It should be noted that it is also possible to load the drugs in the adhesive layer by the electrospinning technique as previously shown (Stie et al., 2022, International Journal of Molecular Sciences, 1458). Loading of multiple drugs within the foam layer, or in the foam and electrospinning layer is thus also possible, providing an even greater versatility in terms of the types and number of drugs that can be loaded in NFF. Having multiple layers and several methods for their preparation, i.e., freeze-drying, electrospinning and spraying, expands the versatility and usability of the NFF drug delivery system over existing patch technologies.

TABLE 1
An overview of examples 1-5 of drugs/molecules that have been
encapsulated and released from the multi-layered NFF drug delivery
system. N = 1-5, n = 4-8, where N is the number of
foam batches, and n the number of replica analyzed per batch.
The loading efficiency is represented as mean ± standard deviation.

| | Molecular | Theoretical | Loading |
|---|---|---|---|
| | weight | loading | efficiency |
| Drug | (Da) | (μg/mg) | (%) |
| Desmopressin | 1 069.2 | 29.13 | 97 ± 1 |
| | | 107.17 | 117 ± 1 |
| Vitamin B2 (riboflavin) | 376.4 | 25.23 | 138 ± 7 |
| FITC-Dextran | 4 000 | 5.13 | 98 ± 2 |
| FITC-Dextran | 40 000 | 5.13 | 91 ± 9 |
| Clotrimazole | 344.8 | 15.27 | 110 ± 2 |

### NFF Multi-Layered DDS Improves Permeation of Vitamin B2 (Riboflavin) Across Buccal Mucosa Ex Vivo

As aforementioned, NFF could improve the permeation of the therapeutic peptide desmopressin across ex vivo porcine buccal mucosa. To further demonstrate the versatility and usability of NFF, the permeability of the small molecule vitamin B2 (riboflavin) released from NFF was evaluated and compared to the permeation of vitamin B2 (riboflavin) in solution. In general, also the permeation of vitamin B2 (riboflavin) was improved by NFF compared to the control (FIG. 9), thus demonstrating ex vivo the usability of NFF for the oromucosal delivery for both small molecules and biopharmaceuticals.

1. A dosage form for application on mucosae for release of at least one active pharmaceutical ingredient comprising:
an adhesive layer capable of adhering to a mucosae of a human body or an animal body;
a water-repelling backing layer; and
at least one intermediate layer positioned between the adhesive layer and the water-repelling backing layer;
wherein at least one active pharmaceutical ingredient is incorporated in the at least one intermediate layer and/or in the adhesive layer, wherein the at least one intermediate layer is a porous semi-solid or a solid material, and wherein the adhesive layer is made of fibers.

2. The dosage form according to claim 1, wherein the at least one intermediate layer comprises two or more intermediate layers.

3. The dosage form according to claim 1, wherein the porous semi-solid or the solid material of the at least one intermediate layer has been prepared by freeze-drying, vacuum drying, vacuum drying of an aqueous or organic polymer dispersion, or neat polymer.

4. The dosage form according to claim 1, wherein the adhesive layer comprises fibers produced by spinning.

5. The dosage form according to claim 1, wherein the at least one active pharmaceutical ingredient is incorporated in the at least one intermediate layer.

6. The dosage form according to claim 2, wherein the at least one active pharmaceutical ingredient is incorporated in each of the two or more intermediate layers.

7. The dosage form according to claim 1, wherein the at least one active pharmaceutical ingredient is incorporated in the adhesive layer.

8. The dosage form according to claim 1, wherein each one of the at least one intermediate layer is a solid foam intermediate layer and wherein the adhesive layer is an adhesive fiber layer.

9. The dosage form according to claim 8, comprising:
a) an adhesive fiber layer;
a water-repelling backing layer; and
one solid foam intermediate layer comprising the at least one active pharmaceutical ingredient; or
b) an adhesive fiber layer;
a water-repelling backing layer; and
two or more solid foam intermediate layers each comprising the at least one active pharmaceutical ingredient; or
c) an adhesive fiber layer comprising the at least one active pharmaceutical ingredient;
a water-repelling backing layer; and
at least one solid foam intermediate layer.

10. (canceled)

11. (canceled)

12. The dosage form according to claim 1, wherein the adhesive layer, intermediate layer(s), and backing layer comprise one or more hydrophilic and/or hydrophobic polymers and/or gums selected from the group consisting of polyethylene oxide, methyl cellulose, hydroxypropyl cellulose, hydroxyethyl cellulose, carboxymethyl cellulose, methylhydroxyethylcellulose, hydroxypropyl methylcellulose, hydroxyethylcarboxymethyl-cellulose, carboxymethylhydroxyethylcellulose, polyvinylpyrrolidone, chitosan, polyacrylic acid, acacia, polylactic acid, poly(lactic-co-glycolic acid), ethylcellulose, gellan gum, gelatin and polysaccharides, such as xanthan, agar, guar gum, carrageenan agar, alginic acid, polymethacrylate, mixtures thereof, salts thereof, and derivatives thereof.

13. The dosage form according to claim 12, wherein the one or more hydrophilic and/or hydrophobic polymer and/or gums is/are in aqueous or organic dispersion, or as neat polymers with/before fabrication of layers by the process selected from the group consisting of spraying, lyophilisation, spinning, casting, and drying.

14. The dosage form according to claim 1, wherein the adhesive layer comprises a membrane comprising at least one adhesive polymer selected from the group consisting of chitosan, alginate, polyethylene oxide, salts thereof, and derivatives thereof.

15. The dosage form according to claim 1, wherein the at least one active pharmaceutical ingredient is one or more of the group consisting of small molecular entities; peptides, proteins, oligonucleotides, whether prepared by synthesis or expression and/or extraction (biopharmaceuticals), and biological material, and particulate matter.

16. The dosage form according to claim 1, wherein the at least one active pharmaceutical ingredient is selected from the group consisting of anti-inflammatory compounds, hormones, cannabinoids, opioids, immunosuppressive compounds, immune stimulating compounds, antibiotics, antivirals, antifungal, chemotherapeutics, probiotics, prebiotics, nutrients, vitamins, minerals, trace elements, and combinations hereof.

17. The dosage form according to claim 1 for use in administration of an active pharmaceutical ingredient to a human or an animal, wherein the adhesive layer adheres to the mucosa of the human or the animal, and wherein the at least one active pharmaceutical ingredient is released unidirectional in a controlled way to the mucosa of the human or the animal.

18. A method of forming a dosage form comprising:
an adhesive layer capable of adhering to a mucosae of a human body or an animal body;
a water-repelling backing layer; and
at least one intermediate layer positioned between the adhesive layer and the water-repelling backing layer;
wherein at least one active pharmaceutical ingredient is incorporated in the at least one intermediate layer and/or in the adhesive layer, wherein the at least one intermediate layer is a porous semi-solid or a solid material, and wherein the adhesive layer is made of fibers, wherein the method comprises:
forming an adhesive layer which is capable of adhering to the mucosa of the human or the animal body;
forming the at least one intermediate layer, wherein the intermediate layer is a porous semi-solid or solid material; and
covering the intermediate layer with a water-repelling backing layer;
wherein the at least one active pharmaceutical ingredient is incorporated in the at least one intermediate layer and/or in the adhesive layer.

19. The method as claimed in claim 18, wherein the porous semi-solid or solid material is formed by freeze-drying, or vacuum drying of an aqueous or organic polymer dispersion, or neat polymer.

20. The method according to claim 18, wherein the adhesive layer made of fibers is formed by spinning.

21. The method according to claim 20, wherein the fibers of the adhesive layer are produced from an aqueous polymer dispersion and/or an organic solvent or the neat polymer.

22. The method according to claim 18, wherein the covering of the at least one intermediate layer with a water-repelling backing layer is done by spraying a polymer dispersion and/or by film formation by heating or light irradiation, by adhering a preformed water-repelling backing layer by wetting or heat, or by freeze-drying or vacuum drying of the polymer dispersion onto the backing layer prepared by casting.
